(19) United States
(12) Patent Application Publication
(10) Pub. No.: US 2013/0178765 A1
(43) Pub. Date: Jul. 11, 2013

(54) ULTRASOUND NEUROMODULATION OF SPINAL CORD

(71) Applicant: David J. Mishelevich, Playa del Rey, CA (US)

(72) Inventor: David J. Mishelevich, Playa del Rey, CA (US)

(21) Appl. No.: 13/689,178

(22) Filed: Nov. 29, 2012

Related U.S. Application Data
(60) Provisional application No. 61/564,856, filed on Nov. 29, 2011.

(51) Int. Cl. A61N 7/00

(52) U.S. Cl. CPC A61N 7/00
USPC 601/2

(57) ABSTRACT

Methods and systems for non-invasive neuromodulation of the spinal cord utilize a transducer to deliver pulsed ultrasound energy to up regulate or down regulate neural targets for the treatment of pain and other disease conditions. The systems provide control of direction of the energy emission, intensity, frequency, pulse duration, pulse pattern, mechanical perturbation, and phase/intensity relationships to achieve up regulation and/or down regulation. One embodiment focuses an elongate tubular ultrasound beam which can be aligned with a target region of the spinal cord.

### CROSS REFERENCE TO RELATED APPLICATIONS

This patent application claims priority to Provisional Application No. 61/564,856, entitled “ULTRASOUND NEUROMODULATION OF THE SPINAL CORD,” filed Nov. 29, 2011, the entire contents of which is incorporated herein by reference.

### INCORPORATION BY REFERENCE

All publications, including patents and patent applications, mentioned in this specification are herein incorporated by reference in their entirety to the same extent as if each individual publication was specifically and individually cited to he incorporated by reference.

### FIELD OF THE INVENTION

The present invention relates generally to methods and systems for neuromodulation and more particularly to methods and systems for neuromodulation of a patient's spinal cord for treatment of pain and other conditions.

### BACKGROUND OF THE INVENTION

It has been demonstrated that focused ultrasound directed at neural structures can stimulate those structures. If neural activity is increased or excited, the neural structure is up regulated. If neural activity is decreased or inhibited, the neural structure is down regulated. Neural structures are usually assembled in circuits. For example, nuclei and tracts connecting them make up a circuit. The effect of ultrasound on neural circuits is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise above 50 degrees C. or tissue will be destroyed (e.g., 56 degrees C. for one second). The second mechanism is mechanical perturbation. An explanation for this has been provided by Tyler et al. from Arizona State University (Tyler, W. J, Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as low intensity low frequency ultrasound (LILFU). Tyler et al. used bursts of ultrasound at frequencies between 0.44 and 0.67 MHz, lower than the frequencies used in imaging. Their device delivered 23 milliwatts per square centimeter of brain a fraction of the roughly 180 mW/cm² upper limit established by the U.S. Food and. Drug Administration (FDA) for womb-scanning sonograms; thus such devices should he safe to use on patients. Ultrasound impact to open calcium channels has also been suggested. This approach is further described in WO 2010/009141 and WO 2011/057028. Of course, the power needed for stimulation of the spinal cord is significantly less than needed for deep-brain neuromodulation. Alternative mechanisms for the effects of ultrasound may be discovered as well. In fact, multiple mechanisms may come into play, but, in any case, this would not effect this invention.

Other approaches for delivering focused ultrasound have also been proposed. Hvstritsky (U.S. Pat. No. 7,283,861) describes the delivery of focused ultrasound pulses (FUP) produced by multiple ultrasound transducers (said preferably to number in the range of 300 to 1000) arranged in a cap place over the skull to provide a multi-beam output. These transducers are coordinated by a computer and used in conjunction with an imaging system. The user interacts with the computer to direct the FUP to the desired point in the brain, sees where the stimulation actually occurred by viewing the image, and can adjust the position of the FUP accordingly. A position of focus is obtained by adjusting the phases and amplitudes of the ultrasound. The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 500 Hz.) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging

US 2009/0112133 describes an alternative approach in which modifications of neural transmission patterns between neural structures and/or regions may be achieved using ultrasound (including use of a curved transducer and a lens) or RF. The impact of long-term potentiation (LTP) and long-term depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm whereas TMS can be focused to 1 cm at best.

Methods and systems for delivering ultrasound energy to neural targets with mechanical perturbation are described in applicant's earlier patent publications including US2011/0208094; US2011/0190668; and US2011/0270138.

The treatment of neuropathic pain has been demonstrated using electrical spinal cord stimulation (SCS) using electrodes to suppress hyperexcitability of the neurons via alteration of dorsal horn neurochemistry including the release of serotonin, Substance P, and GABA. For treatment of ischemic pain, it has been suggested that the oxygen supply may berestored via sympathetic stimulation and/or vasodilation.

### SUMMARY OF THE INVENTION

One purpose of this invention to provide methods and systems for neuromodulation of the spinal cord to treat certain types of pain. Such applicable conditions are non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pant (including that found in irritable bowel syndrome), and refractory ischemic pain (e.g., angina). For pain treatment, the ultrasound energy is targeted to the dorsal column of the spinal cord. In certain embodiments which employ ultrasound neuromodulation, pain is replaced by tingling parathesia. In certain embodiments ultrasound neuromodulation stimulates pain inhibition pathways and can produce acute or long-term effects. The latter can be achieved through long-term potentiation (LTP) or long-term depression (LTD) via training.

The ultrasound energy may be directed at the same target regions in the spinal cord that have been targeted by electrical spinal cord stimulation. For example, for sciatic pain (typically dermatome level L5-S1), ultrasound stimulation can be directed at T10. For angina, the ultrasound energy can be directed at the lower cervical and upper thoracic region. For the abdominal/visceral pain, the ultrasound can be directed at T5-7. Acute and chronic vasculitis can be treated and associated pain by stimulation of regions of the spinal cord as taught in the literature with regard to SCS (Raso, R. and T. Deer, “Spinal Cord Stimulation in the Treatment of Acute and Chronic Vasculitis: Clinical Discussion and Synopsis of the Literature,” Neuromodulation 14:225-228, 2011).

In addition to pain treatment, ultrasound treatment of the spinal cord according to the present invention can treat other conditions such as refractory overactive bladder (e.g., urgency/frequency and urge incontinence) via sacral neuromodulation (Kacker R. and A. K. Das, “Selection of ideal candidates for neuromodulation in refractory overactive bladder,” Current Urology Reports, 11(6):372-378, November 2010) or stimulation of a neurogenic bladder to cause emptying.

Another clinical application of the ultrasound treatments of the present invention comprises the reduction of pain caused by functional bowel disorders such as GI visceral pain and irritable bowel syndrome where myeloperoxidase activity is decreased, inflammation is suppressed, and abdominal relax contractions are inhibited. Suitable target regions in the spinal cord are taught in U.S. Pat. No. 7,251,529.

The present invention further includes control of focus, direction, intensity, frequency (carrier frequency and/or amplitude modulation frequency), pulse duration, pulse pattern, and phase/intensity relationships of the ultrasound energy as well as accomplishing up-regulation and/or down-regulation of the target region of the spinal cord. Use of ancillary monitoring or imaging to provide feedback is optional. In embodiments where concurrent imaging is performed, the device of the invention may be constructed of non-ferrous material.

The specific targets and/or whether the given target is up regulated or down regulated, can depend on the individual patient and relationships of up regulation and down regulation among targets, and the patterns of stimulation applied to the targets. While ultrasound can be focused down to a diameter on the order of one to a few millimeters (depending on the frequency), whether such a tight focus is required depends on the conformation of the neural target.

In a first aspect of the present invention, a method to alleviate a disease condition comprises aiming at least one ultrasound transducer at a target region of a patient's spinal cord. Pulsed power is applied to the transducer to deliver pulsed ultrasound energy to the target region. The disease condition is usually pain where the target region in the spinal cord is typically within the dorsal column. In specific embodiments, the ultrasound transducer is configured to deliver ultrasound energy having an elongated tubular focus aligned with an axis of the spinal cord. Optionally, the ultrasound will be focused where the focus may optionally be mechanically perturbed to enhanced the stimulatory effect of the energy.

In other specific aspects of the methods of the present invention, aiming may comprise aiming a plurality of ultrasonic transducers whose beams intersect at or over the target region. The aiming may alternatively comprise steering a phased array to scan a beam along a segment of the spinal cord. The pulsed ultrasound, may provide up-regulation of the target region, e.g. where the ultrasound energy has a modulation frequency of 500 Hz or higher, a pulse duration from 0.1 msec to 20 msec, and a repetition frequency of 2 Hz or higher. Alternatively, the pulsed ultrasound may provide down-regulation of the target region, e.g. where the ultrasound energy has a modulation frequency of 500 Hz or less, a pulse duration from 0.1 msec to 20 msec, and a repetition frequency of 2 Hz or less. In still other specific aspects of the methods of the present invention, the ultrasound energy provides acute, long-term potentiation of the target region. Alternatively, the ultrasound energy may provide acute, long-term depression of the target region. The methods may further comprise the patient providing feedback as well providing a concurrent therapy selected from the group consisting of transcranial magnetic stimulation (TMS), electrical spinal cord stimulation (SCS), and medication.

The pain disease condition being treated may be selected from the group consisting of non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), refractory pain due to ischemic (e.g, angina), acute vasculitis, chronic vasculitis, hyperactive bladder, and neurogenic bladder.

Dorsal lateral lower motor neurons are associated with the lateral corticospinal tract. Ventromedial lower motor neurons are associated with the anterior corticospinal tract. In an embodiment of the current invention, ultrasound neuromodulation exciting of those motor neurons or their associated tracts results in contractions of the connected muscles. Thus in some embodiments, the ultrasound energy can be employed to restore motor neuron function.

In a second aspect of the present invention, apparatus for delivering ultrasound energy to a target region of a patient's spinal cord comprises an ultrasound transducer assembly and control circuitry and/or supporting structure for delivering ultrasound energy from the transducer assembly to the target region of the spinal cord. The ultrasound energy delivery control circuitry and/or supporting structure preferably focuses the ultrasound along a tubular target region aligned with an axis of the spinal cord. The transducer may comprise an elongated transducer having an active surface formed over a partial tubular groove for focusing the ultrasound energy along the tubular target region. The transducer body may consist of a single piezoelectric element or alternatively may include an array of individual transducer elements, e.g. arranged as a phased array for focusing the energy in the tubular focus or other desired focus geometry. The ultrasound transducer may be supported or controlled to mechanically perturb the ultrasound energy, e.g. the ultrasound transducers may be moved to apply mechanical perturbations radially and/or axially. In specifically preferred aspects, the ultrasound transducer and the energy delivery means may be configured to deliver ultrasound energy to the patient's dorsal column for the treatment of pain.

In still other aspects of the present invention, the ultrasound transducer and the energy delivery struduremay be configured to deliver ultrasound energy to up-regulate or down-regulate the target region. The ultrasound transducer and the energy delivery control and support structure may be configured to deliver ultrasound energy with a modulation frequency of 500 Hz or less, a pulse duration from 0.1 msec to 20 msec, and a repetition frequency of 2 Hz or less to down regulate the target region. Alternatively the ultrasound transducer and the energy delivery control and support structure may be configured to deliver ultrasound energy with a modulation frequency of 500 Hz or higher, a pulse duration from 0.1 msec to 20 msec, and a repetition frequency of 2 Hz or higher to up regulate the target region.

Apparatus of the present invention may be further configured to deliver ultrasound energy that provides long-term potentiation of the target region long-term depression of the target region. Apparatus may further comprise a patient feedback mechanism and may further be combined with system elements for delivering transcranial magnetic stimulation (TMS), electrical spinal cord stimulation (SCS).

### BRIEF DESCRIPTION OF THE DRAWINGS

FIG. 1 shows ultrasound-transducer targeting of the spinal cord from the perspective view of the spinal column.

FIG. 2 shows ultrasound-transducer targeting of the spinal cord from the cross-section view of the spinal column.

FIGS. 3A-3C illustrate shaping of the ultrasound field.

FIGS. 4A and 4B show the mechanism for mechanical perturbation and examples the resultant ultrasound field shapes.

FIG. 5 shows a block diagram of the control circuit.

FIG. 6 illustrates a block diagram for a mechanism providing patient feedback for adjustment of the characteristics of the neuromodulation.

### DETAILED DESCRIPTION OF THE INVENTION

It is the purpose of this invention to provide methods and systems and methods for neuromodulation of the spinal cord to treat certain types of pain. Such pain conditions include non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), and refractory pain due to ischemia (e.g. angina). In certain embodiments of the present invention, pain is replaced by tingling parathesias. In certain embodiments of the present invention, ultrasound neuromodulation stimulates pain inhibition pathways and can produce acute or long-term effects. The latter occur through long-term depression (LTD) or long-term potentiation (LTP) via training. Acute and chronic vasculitis can be treated as well as associated pain. In addition, sacral neuromodulation can be employed for the treatment of hyperactive bladder as well as to stimulate emptying of a neurogenic bladder. Included is control of direction of the energy emission, intensity, frequency (carrier frequency and/or neuromodulation frequency), pulse duration, pulse pattern, and phase/intensity relationships to targeting and accomplishing up-regulation and/or down-regulation.

Target regions in the spinal cord which can be treated using the ultrasound neuromodulation protocols of the present invention comprise the same locations targeted by electrical SCS electrodes for the same conditions being treated, e.g., a lower cervical-upper thoracic target region for angina, a T5-7 target region for abdominal/visceral pain, and a T10 target region for sciatic pain. Ultrasound neuromodulation in accordance with the present invention can stimulate pain inhibition pathways which in turn can produce acute and/or long-term effects. Other clinical applications of ultrasound neuromodulation of the spinal cord include non-invasive assessment of neuromoduation at a particular target region in a patient's spinal cord prior to implanting an electrode for electrical spinal cord stimulation for pain or other conditions.

The stimulation frequency for inhibition may be lower than 500 Hz (depending on condition and patient). The stimulation frequency for excitation may be above 500 Hz, typically being in the range of 500 Hz to 5 MHz. In this invention, the ultrasound acoustic frequency is in range of 0.3 MHz to 0.8 MHz with power generally applied less than 60 mW/cm² usually less than 21 mW/cm², often less than 10 mW/cm². The acoustic frequency is modulated at the lower rate to impact the neuronal structures as desired (e.g., 300 Hz for inhibition (down-regulation) or 1 kHz for excitation (up-regulation). The modulation frequency (superimposed on the carrier frequency of say 0.5 MHz or similar) may be divided into pulses 0.1 to 20 msec repeated at frequencies of 2 Hz or lower for down regulation and higher than 2 Hz for up regulation) although this will be both patient and condition specific. The number of ultrasound transducers can vary between one and 500.

The lower size boundary of the spot or line width of the focused ultrasound energy will depend on the ultrasonic frequency, with higher frequencies generally corresponding to smaller spots or widths. Ultrasound-based neuromodulation operates preferentially at low frequencies relative to say imaging applications so there is less resolution. A suitable one-inch diameter ultrasound transducer having a focal length of two inches that operates with a 0.4 Mhz excitation frequency and will deliver a focused spot with a diameter (6 dB) of 0.29 inches is available from Keramos-Etalon. Typically, the spot size will be in the range of 0.1 inch to 0.6 inch depending on the specific indication and patient. A larger spot can be obtained with a one-inch diameter ultrasound transducer with a focal length of 3 inch which operates at 0.4 MHz excitation and will deliver a focused spot with a diameter (6 dB) of 0.51 inch. Even though the target is relatively superficial, the transducer can be moved back in the holder to allow a longer focal length. Other embodiments are applicable as well, including different transducer diameters, different frequencies, and different focal lengths. Other ultrasound transducer manufacturers include Blatek and Imasonic. In an alternative embodiment, focus can be deemphasized or eliminated with a smaller ultrasound transducer diameter with a shorter longitudinal dimension, if desired, as well. Ultrasound conduction medium will usually be provided to fill the space between the transducer and the patient's skin.

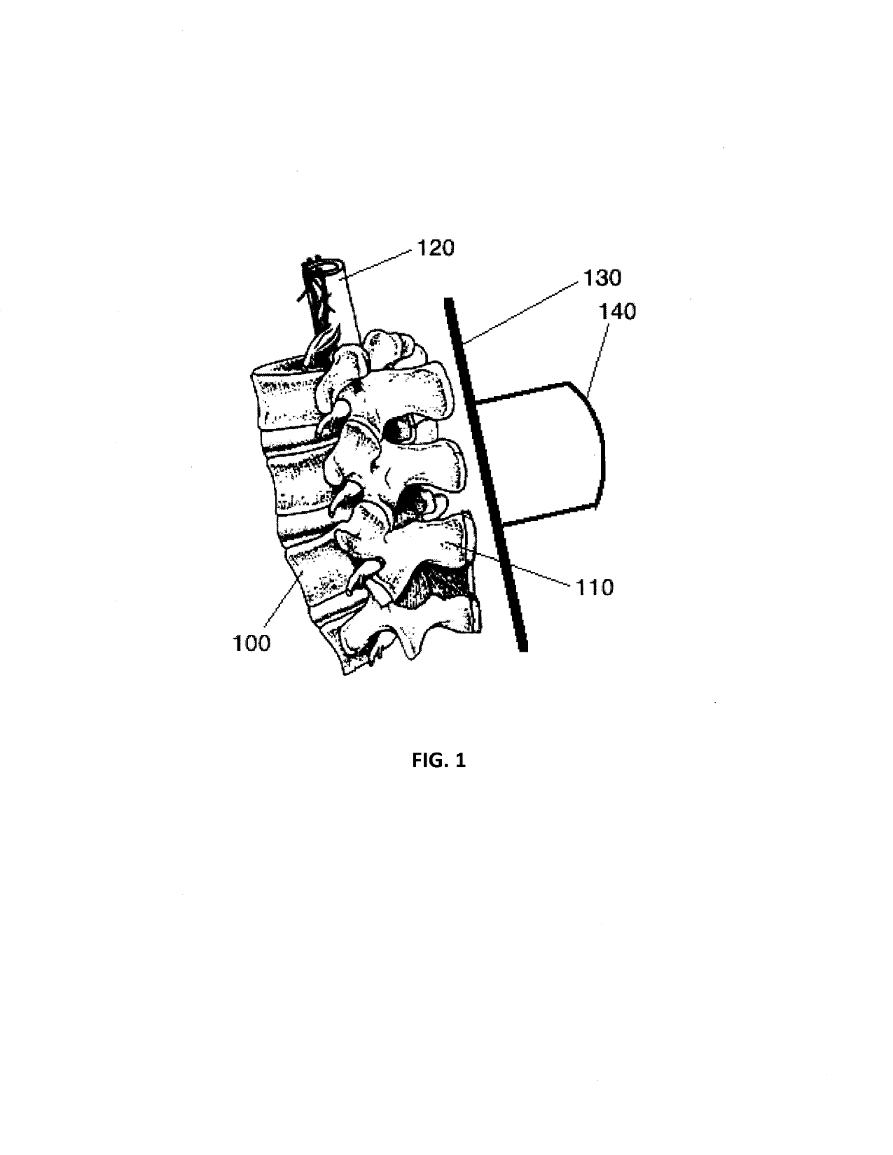

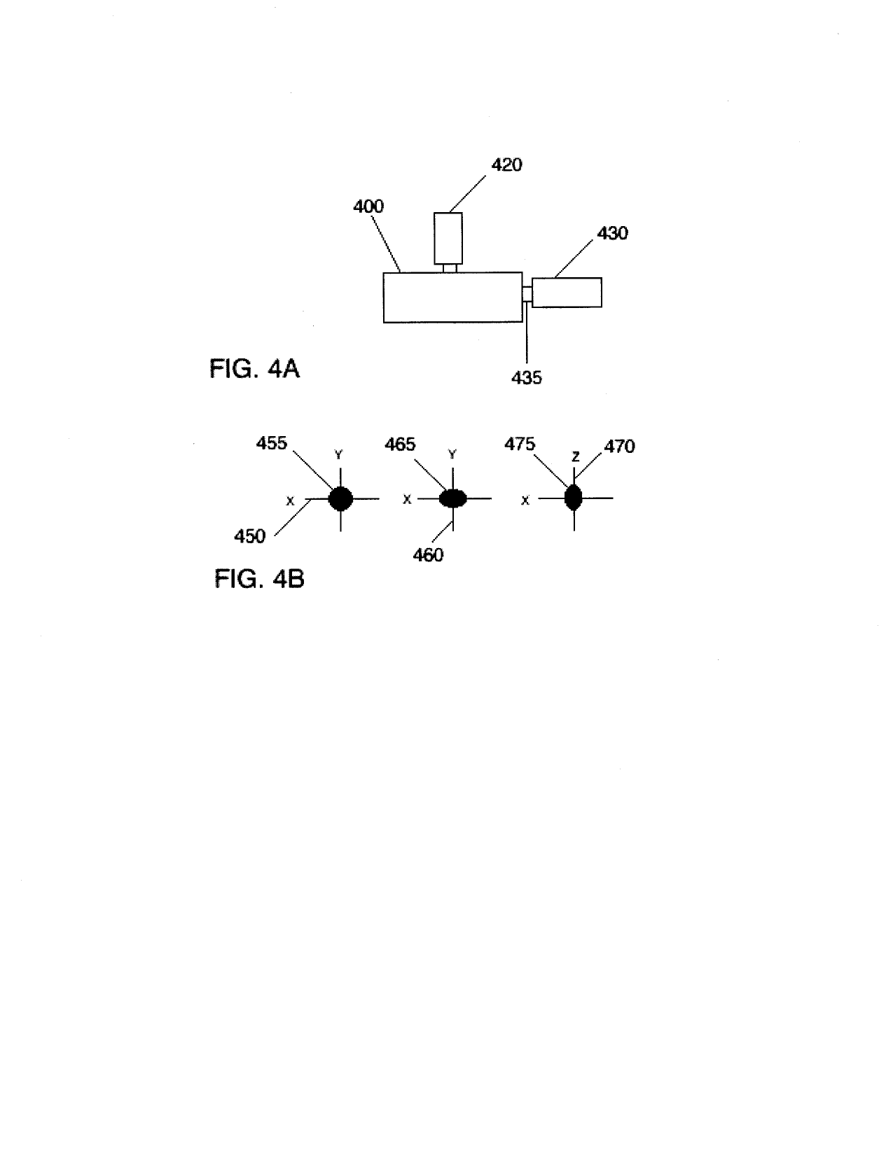

FIG. 1 shows spinal column with vertebrae 100 and spinal process 110 containing spinal cord 120 covered by skin 130. Spinal cord 120 is neuromodulated by ultrasound transducer 140. For ultrasound to be effectively transmitted to and through the skin and to target spinal-cord target, coupling must be put into place. A layer of ultrasound transmission gel (not shown) is placed between the face of the Ultrasound transducer and the skin over the target. If filling of additional space (e.g., within the transducer housing or between the transducer face and the skin), an ultrasound transmission medium (for example Dermasol from California Medical Innovations) can be used. In another embodiment, multiple ultrasound transducers whose beams intersect at that target replace an individual ultrasound transducer for that target. Transducers can be placed on both sides of the spinous processes to direct beams inwardly to integrate along the spinal cord or can be located on one side only and focused medially to target the spinal cord. In still another embodiment, mechanical perturbations are applied radially or axially to move the ultrasound transducers, as discussed below with reference to FIGS. 4A and 4B.

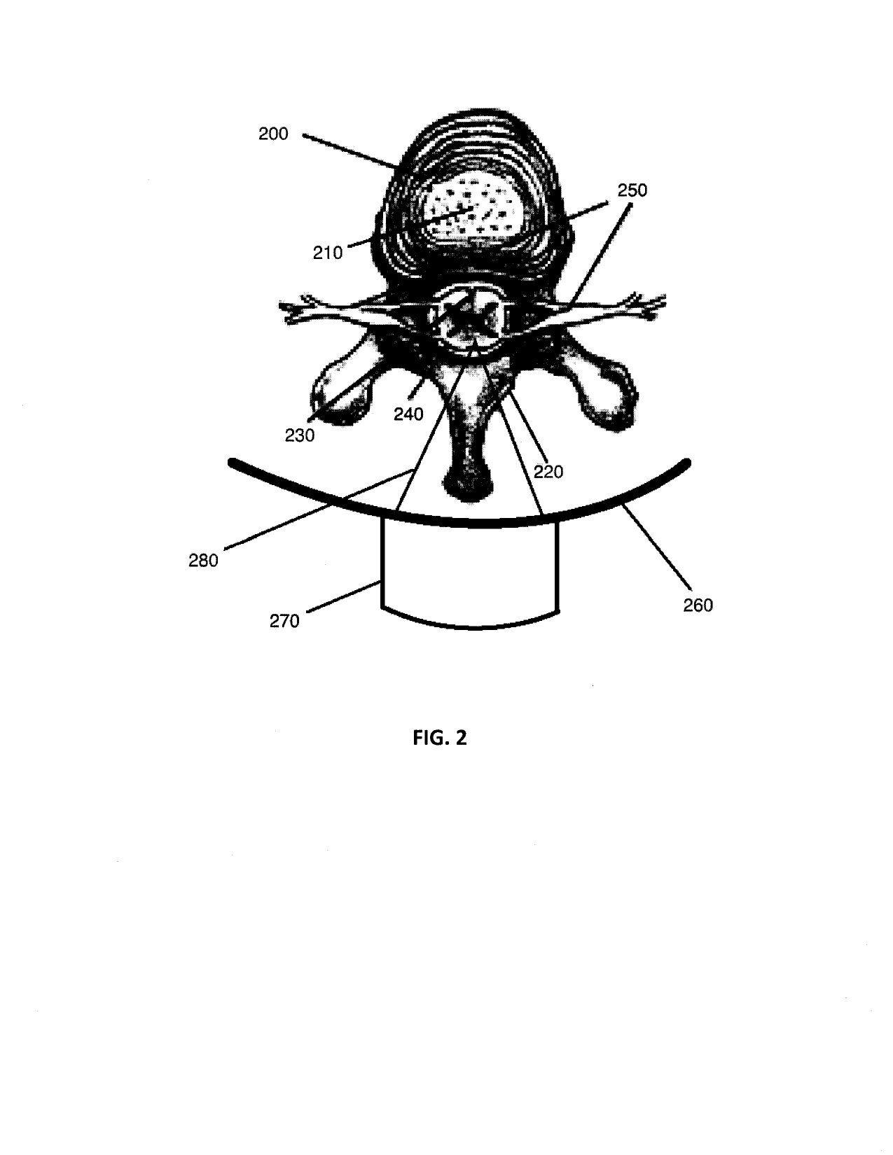

FIG. 2 shows a cross section of the spinal column and spinal cord. Vertebrae disc 200 with its nucleus pulposus 210 with other bony structures such as the lamina 220 surrounds the dura 240 surrounding spinal cord 230 with its spinal nerve roots 250. Ultrasound transducer 270 is pressed against skin 260 and generates ultrasound beam 280 that neuromodulates nerves within spinal cord 230. Bilateral neuromodulation of spinal cord 230 can be performed. For ultrasound to be effectively transmitted to and through the skin and to target spinal-cord target, coupling must be put into place. A layer of ultrasound transmission gel (not shown) is placed between the face of the ultrasound transducer and the skin over the target. If filling of additional space (e.g., within the transducer housing), an ultrasound transmission medium (for example Dermasol from California Medical Innovations) can be used. In another embodiment, multiple ultrasound transducers whose beams intersect at that target replace an individual ultrasound transducer for that target. In still another embodiment, mechanical perturbations are applied radially or axially to move the ultrasound transducers (FIGS. 4A-4B).

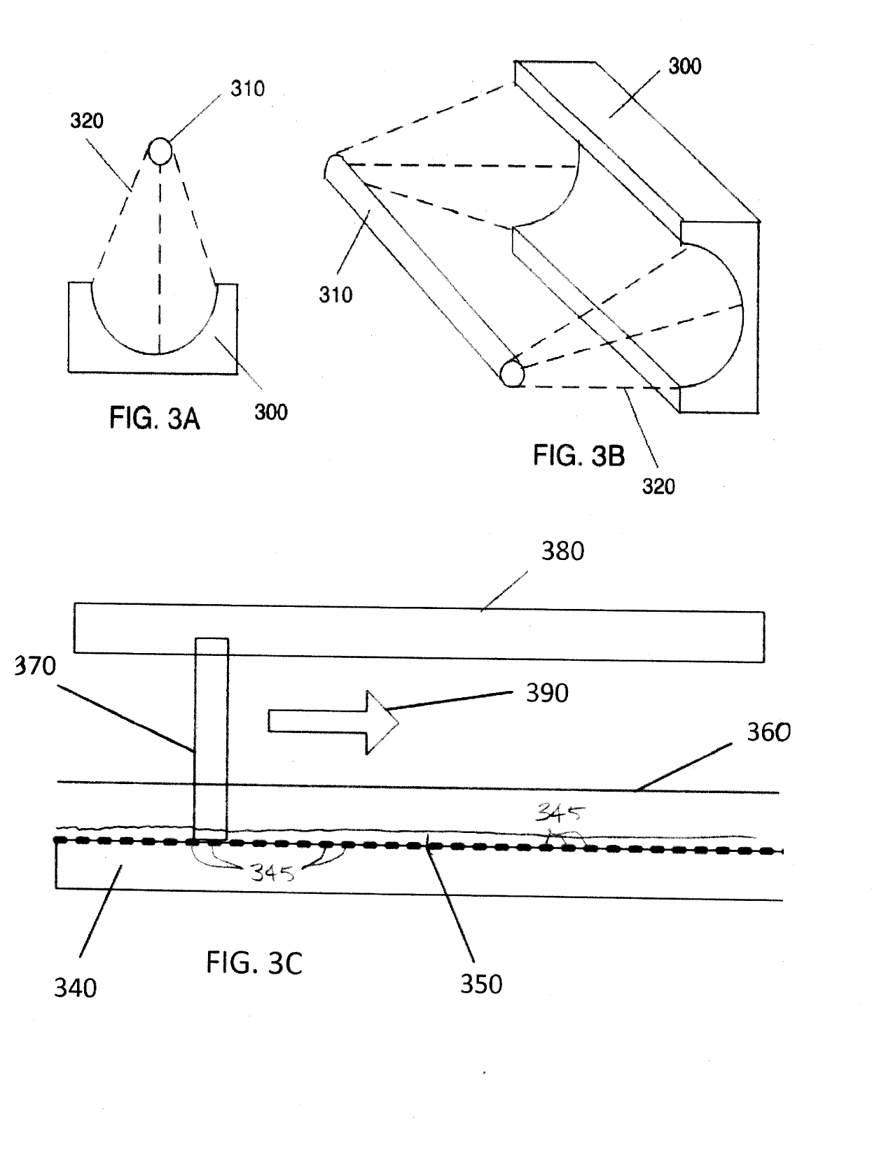

FIGS. 3A and B show an exemplary ultrasound transducer assembly 300 that may be a shaped piezoelectric transducer body or may comprise an array of individual transducer elements configured to produce an elongated tubular (e.g. pencil-shaped) focused field 310. Such a transducer assembly is applied to stimulate an elongated target such as the spinal cord. In alternative embodiments, a spot focused ultrasonic energy beam may be over any portion of the length of the spinal cord to target specific target regions. In both cases, it is possible to determine over what length of a target region that the ultrasound is to be applied. For example, one could apply ultrasound to only a selected portion of the spinal cord. In FIG. 3A, an end view of the array is shown with curved-cross section ultrasonic array 300 forming a sound field 320 focused on target 310. FIG. 3B shows the same array in a side view, again with ultrasound array 300, target 310, and focused field 320.

FIG. 3C shows a linear ultrasound phased array 340 which can “steer” an ultrasound beam 370 by changing the phase/intensity relationships of a plurality of individual transducer elements 345. In this way, ultrasound beams can be moved (steered) and focused without physically displacing the array 340 of transducers 345. The beam direction can be directed at angles which are perpendicular or non-perpendicular to the surface of the transducer array, and beam direction is thus not restricted to being aimed perpendicularly from the face of the transducer or array. In FIG. 3C, the transducer array 340 is flat and emits ultrasound conducted by a conducting gel layer 350 providing the physical interface to skin over spinal column 360. The beam 370 of ultrasound energy moves linearly from left to right as shown by arrow 390 so it moves its focus along spinal cord target 380. Transducers can be place on either side of the spinous processes or placed on one side and aimed medially. In still another embodiment, mechanical perturbations may be applied to move the ultrasound transducers as covered in FIG. 4, for example, to increase ultrasound field depth. In another embodiment, the surface of the transducer array is not flat but curved.

Transducer array assemblies of this type may be supplied with custom specifications by Imasonic in France (e.g, large 2D High Intensity Focused Ultrasound (HIFU) hemispheric array transducer; and Fleury G., Berriet, R., Le Baron, O., and B. Huguenin, “New piezocomposite transducers for therapeutic ultrasound,” 2^{nd}International Symposium on Therapeutic Ultrasound—Seattle—31/07—02/08/02), typically with numbers of ultrasound transducers of 300 or more. Keramos-Etalon in the United States is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

FIGS. 4A and 4B show the mechanism for mechanical perturbation of the ultrasound transducer. In FIG. 4A illustrating a plan view with mechanical actuators 420 and 430 moving ultrasound transducer 400 in and out and left respectively. Actuator rod 435 provides the mechanical interface between mechanical actuator 430 and ultrasound transducer 400 as an example. Not shown is an equivalent mechanical actuator moving ultrasound transducer 400 along an axis perpendicular to the page. Such mechanical actuators can have alternative configurations such as motors, vibrators, solenoids, magnetostrictive, electrorestrictive ceramic and shape memory alloys. Piezo-actuators such as those provided by DSM can have very fine motions of 0.1% length change. FIG. 4B shows effects on the focused ultrasound modulation focused at the target. The axes are 450 (x,y), 460 (x,y,) and 470 (x,z). As demonstrated on 450 the excursions along x and y from 430 and 420 are equal so the resultant pattern is a circle. As demonstrated on 460 the excursion due to 430 is greater than that if 420 so the resultant pattern is longer along the x axis than the y axis. As demonstrated on 470, the excursion is longer along the z axis than the x axis to the resultant pattern is long along the z axis than the x axis. Not shown is the inclusion of the impacts of actuation along the axis perpendicular to the page. In each case, the pattern would be matched to the shape of the target of the modulation. For the transducer arrangement shown in FIGS. 3A and 3B, depth can be added, to the length and width which are produced.

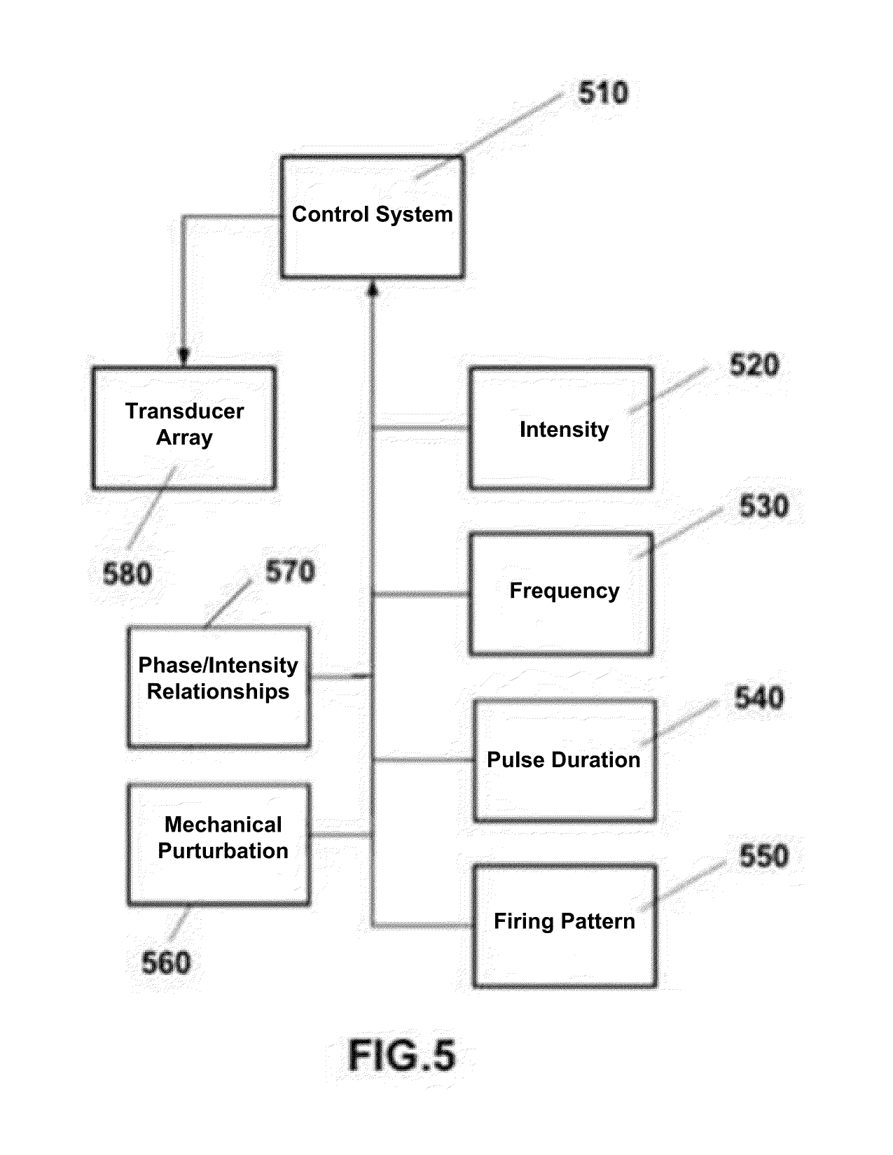

FIG. 5 shows an embodiment of a control circuit. The positioning and emission characteristics of transducer array 580 are controlled by control system 510 with control input with neuromodulation characteristics determined by settings of intensity 520, frequency (including carrier frequency) 530, pulse duration 540, firing pattern 550, mechanical perturbation 560, and phase intensity relationships 570 for beam steering and focusing on neural targets.

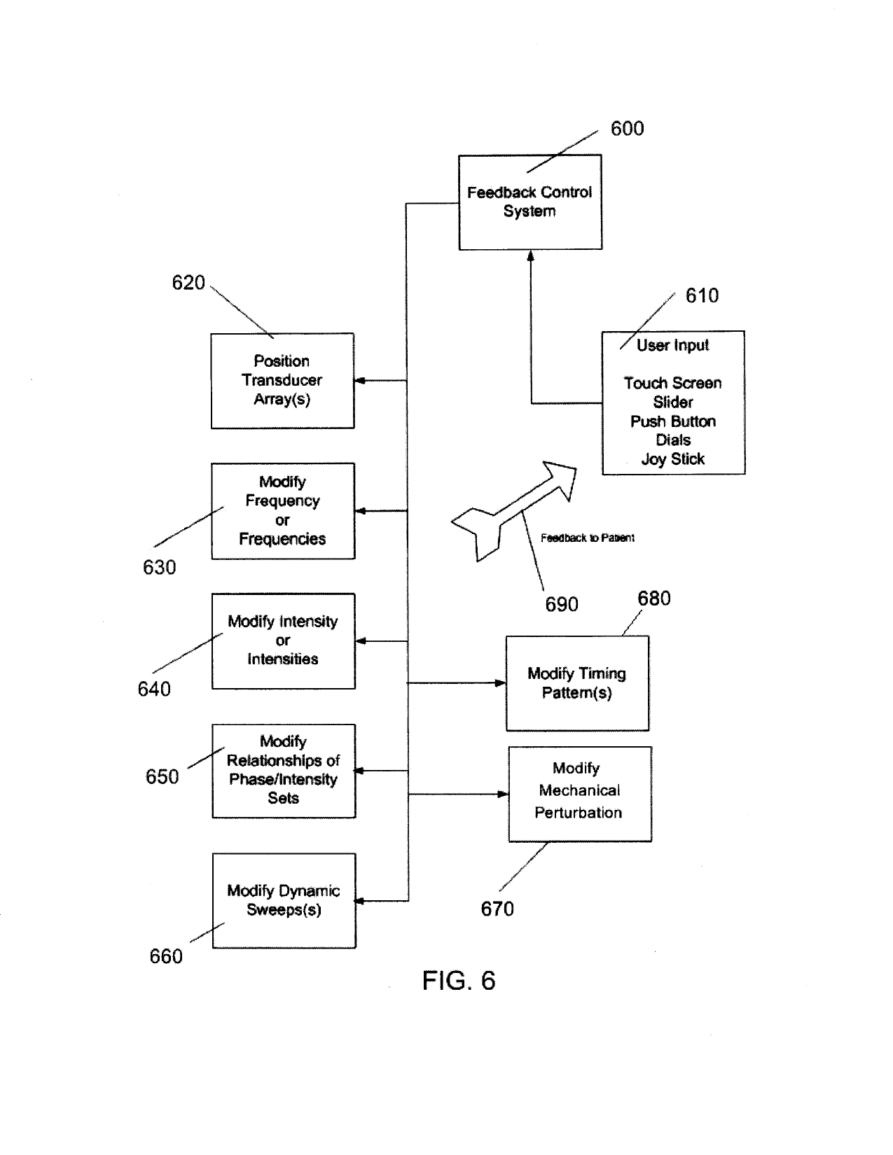

The operator can set the variables for the ultrasound neuromodulation or the patient can do so. FIG. 6 shows the basic feedback circuit. Feedback Control System 600 receives its input from User Input 610 and provides control output for positioning ultrasound transducer arrays 620, modifying pulse frequency or frequencies 630, modifying intensity or intensities 640, modifying relationships of phase/intensity sets 650 for focusing including spot positioning via beam steering, modifying dynamic sweep patterns 660, modifying mechanical perturbation 670, and/or modifying timing patterns 680. Feedback to the patient 690 occurs with what is the physiological effect on the patient (for example increase or decrease in pain or decrease or increase on tremor). User Input 610 can be provided via a touch screen, slider, dials, joystick, or other suitable means. Often the user can be the best judge what alterations of what changes in neuromodulation will be most helpful, either changing one variable at a time or multiple variables. One example of patient control is the patient (e.g., one with a transected spinal cord) directly turning on the neuromodulation to empty a neurogenic bladder.

In still other embodiments, other energy sources are used, in combination with or substituted for ultrasound transducers that are selected from the group consisting of Transcranial Magnetic Stimulation (TMS), Spinal Cord Stimulation (SCS), and medications.

The invention allows stimulation adjustments in variables such as, but not limited to, direction of the energy emission, intensity, frequency (carrier frequency and/or neuromodulation frequency), pulse duration, pulse pattern, and phase/intensity relationships to targeting and accomplishing up-regulation and/or down-regulation, dynamic sweeps, mechanical perturbation, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method to alleviate a disease condition, the method comprising:
aiming at least one ultrasound transducer at a target region of a patient's spinal cord, and
applying pulsed power to the transducer to deliver pulsed ultrasound energy to the target region.

2. The method of claim 1, wherein the disease condition is pain and the target region comprises the dorsal column.

3. The method of claim 1, wherein the ultrasound transducer is configured to deliver ultrasound energy having an elongated tubular focus aligned with an axis of the spinal cord.

4. The method of claim 1, further comprising mechanically perturbing the ultrasound energy.

5. The method of claim 1, wherein aiming comprises aiming a plurality of ultrasonic transducers whose beams intersect at or over the target region.

6. The method of claim 1, wherein aiming comprises steering an ultrasound beam from a phased ultrasound array.

7. The method of claim 1, wherein the pulsed ultrasound provides up-regulation of the target region.

8. The method of claim 5, wherein the ultrasound energy has a modulation frequency of 500 Hz or higher, a pulse duration from 0.1 msec to 20 msec, and a repetition frequency of 2 Hz or higher.

9. The method of claim 1, wherein the pulsed ultrasound provides down-regulation of the target region.

10. The method of claim 6, wherein the ultrasound energy has a modulation frequency of 500 Hz or less, a pulse duration from 0.1 msec to 20 msec, and a repetition frequency of 2 Hz or less.

11. The method of claim 1, wherein ultrasound energy provides acute, long-term potentiation of the target region.

12. The method of claim 1, wherein ultrasound energy provides acute, long-term depression of the target region.

13. The method of claim 1, wherein the disease treated is selected from the group consisting of non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, limctional bowel disorder pain (including that found in irritable bowel syndrome), refractory pain due to ischemic (e.g, angina), acute vasculitis, chronic vasculitis, hyperactive bladder, and neurogenic bladder.

14. The method of claim 1, wherein the pulsed ultrasound energy produces motor neurons.

15. The method of claim 1, further comprising the patient providing feedback.

16. The method of claim 1, further comprising providing a concurrent therapy selected from the group consisting of transcranial magnetic stimulation (TMS), electrical spinal cord stimulation (SCS), and medication.

17. Apparatus for delivering ultrasound energy to a target region of a patient's spinal cord, said apparatus comprising:
an ultrasound transducer assembly, and
means for delivering ultrasound energy from the transducer assembly to the target region of the spinal cord.

18. Apparatus as in claim 17, wherein the ultrasound energy deliver means focuses the ultrasound along a tubular target region aligned with an axis of the spinal cord.

19. Apparatus as in claim 18, wherein the transducer comprises an elongated transducer having an active surface formed over a partial tubular groove for focusing the ultrasound energy along the tubular target region.

20. Apparatus as in claim 19, wherein the transducer body consists of a single piezoelectric element.

21. Apparatus as in claim 17, wherein the transducer comprises a phased array having a length and width which configure to a segment of a spinal cord.

22. Apparatus as in claim 17, wherein the means for delivering ultrasound energy from the transducer assembly to the target region of the spinal cord is configured to mechanically perturb the ultrasound energy.

23. Apparatus as in claim 22, wherein the ultrasound transducers are moved to apply mechanical perturbations radially and/or axially.

24. Apparatus as in claim 17, wherein the ultrasound transducer and the energy delivery means are configured to deliver ultrasound energy to the patient's dorsal column for the treatment of pain.

25. Apparatus as in claim 17, wherein the ultrasound transducer and the energy delivery means are configured to deliver ultrasound energy to up-regulate the target region.

26. Apparatus as in claim 17, wherein the ultrasound transducer and the energy delivery means are configured to deliver ultrasound energy to down-regulate the target region.

27. Apparatus as in claim 17, wherein the ultrasound transducer and the energy delivery means are configured to deliver ultrasound energy with a modulation frequency of 500 Hz or less, a pulse duration from 0.1 msec to 20 msec, and a repetition frequency of 2 Hz or less to down regulate the target region.

28. Apparatus as in claim 17, wherein the ultrasound transducer and the energy delivery means are configured to deliver ultrasound energy with a modulation frequency of 500 Hz or higher, a pulse duration from 0.1 msec to 20 msec, and a repetition frequency of 2 Hz or higher to up regulate the target region.

29. Apparatus as in claim 17, wherein the ultrasound transducer and the energy delivery means are configured to deliver ultrasound energy which provides long-term potentiation of the target region.

30. Apparatus as in claim 17, wherein the ultrasound transducer and the energy delivery means are configured to deliver ultrasound energy which provides long-term depression of the target region.

31. Apparatus as in claim 17, further comprising a patient feedback mechanism.

32. Apparatus as in claim 17, further comprising means for delivering transcranial magnetic stimulation (TMS) or electrical spinal cord stimulation (SCS).
